(19) United States
(12) Patent Application Publication
(10) Pub. No.: US 2022/0117696 A1
(43) Pub. Date: Apr. 21, 2022

(54) OPTICAL COHERENCE TOMOGRAPHY AUGMENTED REALITY-BASED SURGICAL MICROSCOPE IMAGING SYSTEM AND METHOD

(71) Applicant: SUZHOU INSTITUTE OF BIOMEDICAL ENGINEERING AND TECHNOLOGY, CHINESE ACADEMY OF, SUZHOU (CN)

(72) Inventors: Guohua SHI, SUZHOU (CN); Jinyu FAN, Xiangtan (CN); Yi HE, Chengdu (CN); Lina XING, Changchun (CN); Feng GAO, Yichun (CN)

(21) Appl. No.: 17/646,722

(22) Filed: Jan. 1, 2022

Related U.S. Application Data
(63) Continuation of application No. PCT/CN2019/113695, filed on Oct. 28, 2019.

(30) Foreign Application Priority Data
Jul. 1, 2019 (CN) 201910583463.8

(51) Int. Cl. A61B 90/00; A61B 90/20; A61B 90/30; G02B 21/00; G02B 21/36; G02B 27/14; H04N 13/239; H04N 5/225; H04N 13/302; H04N 5/235; G06T 7/70; G06T 11/00; G06K 9/62; G06T 19/00

(52) U.S. Cl. CPC A61B 90/37; G06T 2207/10056; A61B 90/30; G02B 21/0012; G02B 21/367; G02B 27/141; H04N 13/239; H04N 5/2256; H04N 13/302; H04N 5/2354; G06T 7/70; G06T 11/008; G06K 9/6289; G06T 19/006; A61B 2090/3735; A61B 2090/365; A61B 2090/367; A61B 2090/371; G06T 2207/10101; A61B 90/20

(57) ABSTRACT

An optical coherence tomography (OCT) augmented reality-based surgical microscope imaging system and method. The system has a surgical microscope unit, an OCT unit, a guidance light source, a processing control unit, and a display unit. The surgical microscopic imaging system and method can accurately register and fuse the two-dimensional microscopic image and the OCT three-dimensional image, thereby implementing real-time enhancement of microscopic images in the surgical region, providing more intuitive navigation information for surgery, and realizing intuitive surgical guidance.

### CROSS-REFERENCE TO RELATED APPLICATIONS

This application is a continuation of International Patent Application Number PCT/CN2019/113695, filed on Oct. 28, 2019, which claims the benefit and priority of Chinese Patent Application Number 201910583463.8, filed on Jul. 1, 2019, the disclosures of which are incorporated herein by reference in their entireties.

### TECHNICAL FIELD

The disclosure relates to the technical field of microsurgery imaging and graphic processing, and particularly relates to an Optical Coherence Tomography (OCT) augmented reality-based surgical microscope imaging system.

### BACKGROUND

Modern surgery has required that when a surgical target site is positioned, the physiological trauma on patients should be reduced to the greatest extent to achieve minimally invasive surgery. Image-guided interventional surgery can accurately position the surgical target site, can achieve the characteristics of preoperative planning, intra-operative real-time monitoring navigation, postoperative evaluation on the surgery effect of a surgical region and the like, has the advantages of high accuracy, small trauma and the like, and is an important direction of the modern surgery.

Currently, imaging ranges of optical microscope-based microsurgery such as ophthalmic surgery and neurosurgery are limited to surface two-dimensional imaging, resulting in severe limitation to application of the microsurgery. OCT is a high-resolution high-sensitivity non-contact three-dimensional imaging method, can be used for carrying out imaging on tomography inside tissues and surgical instruments, and is particularly applicable to navigation of fine surgery, so that a Microscope Integrated OCT (MIOCT) surgical navigation system is developed, and meanwhile, development of a high-Speed Sweep-frequency OCT (SS-OCT) technology enables intra-operative application of three-dimensional OCT real-time imaging to become possible. The patent WO2016/172495A1 provides a MIOCT imaging display method in which OCT information and microscope information are simultaneously displayed in an eyepiece. However, in the method, an OCT image is just displayed beside a microscopic image, fused imaging of the OCT image and the microscopic image is not involved, and thus a doctor still needs to carry out subjective matching on the two images during surgery. In order to solve the above problem, the surgical imaging method and equipment need to be improved to acquire more intuitive intra-operative navigation information.

Augmented reality is a technology of fusing the scene in a virtual world on display equipment with the scene in a real world through position and angle refined calculation of a camera video and an image analysis technology. In surgery, the augmented reality technology can fuse a three-dimensional (3D) image such as CT with a real scene so as to implement intuitive surgical guidance. The key of the augmented reality technology is virtual-real registration, i.e., establishment of a coordinate transformation relationship of a virtual image and the real scene, and the difficulty thereof lies in finding positions of the same point in virtual and real coordinate systems, i.e., setting and tracking a datum point. When an artificially placed object is used as the datum point for registration, a matching result with better accuracy can be obtained, but the method possibly causes traumas; and when body surface characteristics are utilized to set the datum point, the additional traumas can be avoided, but when the characteristics are unobvious, the identification effect is poor, resulting in limitation to the application of the method. Therefore, in order to fuse a three-dimensional OCT image serving as a virtual image into the microscopic image, new registration and fusion method and imaging system need to be introduced.

### SUMMARY

The present disclosure is to solve the technical problem of providing an OCT augmented reality-based surgical microscope imaging system and method for the above-mentioned defects in the prior art.

In order to solve the above-mentioned technical problem, the present disclosure adopts the technical solution that an OCT augmented reality-based surgical microscope imaging system includes:

a surgical microscope unit, configured to acquire a two-dimensional microscopic image of a surgical region;

an OCT unit, configured to acquire an OCT three-dimensional image of the surgical region;

a processing control unit, configured to acquire the two-dimensional microscopic image and the OCT three-dimensional image of the surgical region, and an image obtained by fusing the two-dimensional microscopic image and the OCT three-dimensional image of the surgical region; and

a display unit, configured to output and display a result of the processing control unit to carry out navigation for surgery.

The system further includes a guidance light source, which can be captured by the surgical microscope unit and is configured to project, into the surgical region, a guidance light spot synchronized with an OCT scanning light source of the OCT unit.

Preferably, the system further includes a surgical lighting unit and an objective lens, a light splitting unit and an optical zoom unit sequentially arranged along an imaging optical path of the surgical microscope unit;

the surgical lighting unit is configured to provide lighting light for the surgical region, and the lighting light reflected by the surgical region enters the surgical microscope unit after sequentially passing through the objective lens, the light splitting unit and the optical zoom unit so as to implement two-dimensional microscopic imaging of the surgical region; and

light emitted by the guidance light source and the OCT scanning light source of the OCT unit reaches the surgical region after sequentially passing through the light splitting unit and the objective lens, and OCT scanning light reflected by the surgical region backtracks to the OCT unit to implement OCT three-dimensional imaging; and after guidance light reflected by the surgical region passes through the light splitting unit, one portion of the guidance light enters the OCT unit, while the other portion of the guidance light enters the surgical microscope unit.

Preferably, the surgical microscope unit includes imaging lenses and cameras, the imaging lenses include a left imaging lens and a right imaging lens, and the cameras include a left camera and a right camera, wherein the left imaging lens and the left camera correspondingly constitute a left microscopic imaging module, and the right imaging lens and the right camera correspondingly constitute a right microscopic imaging module.

Preferably, the light splitting unit is a dichroic mirror which carries out total reflection on the light of the OCT unit, carries out semi-transmission and semi-reflection on the light of the guidance light source, and carries out total transmission on the light of the surgical lighting unit.

Preferably, the OCT unit includes the OCT scanning light source, a first coupler, a wavelength division multiplexer, a first collimator, a two-dimensional galvanometer scanner, a second collimator, a reflector, a third collimator, a second coupler and a balance detector;

an OCT scanning beam emitted by the OCT scanning light source is split into two paths of light via the first coupler, one path of light is sample light, and the other path of light is reference light;

the guidance light emitted by the guidance light source and the sample light are converged via the wavelength division multiplexer, then pass through the first collimator together to become incident to the two-dimensional galvanometer scanner to be deflected, and then are focused into the surgical region by the objective lens after being reflected by the dichroic mirror;

both the sample light and one portion of guidance light reflected by the surgical region return along an original path after being reflected by the dichroic mirror, and reach one end of the second coupler after passing through the first coupler; the other portion of guidance light reflected by the surgical region transmits through the dichroic mirror after passing through the objective lens, passes through the optical zoom unit, and then respectively passes through the left imaging lens and the right imaging lens to respectively enter the left camera and the right camera;

the reference light emergent after passing through the first coupler sequentially passes through the second collimator, the reflector and the third collimator to reach said one end of the second coupler, and enters the second coupler together with the sample light and said one portion of guidance light that have been reflected by the surgical region and reached said one end of the second coupler, the reference light undergoes interference with the sample light and said one portion of guidance light before being received by the balance detector, and finally, a detection result is output to the processing control unit so as to implement OCT three-dimensional imaging;

after a lighting beam emitted by the surgical lighting unit irradiates the surgical region, the lighting light and the other portion of guidance light reflected by the surgical region transmit through the dichroic mirror, then pass through the optical zoom unit, subsequently enter the left microscopic imaging module and the right microscopic imaging module, and finally, an image signal is output to the processing control unit so as to implement two-dimensional microscopic imaging of the surgical region; and

the processing control unit carries out registration and fusion on the two-dimensional microscopic image and the OCT three-dimensional image of the surgical region, and a fused image is displayed and output by the display unit so as to carry out navigation for surgery.

Preferably, the display unit is a polarized light display screen with a stereoscopic visual effect, and is configured to respectively output an image obtained by fusing the two-dimensional microscopic image from the left microscopic imaging module and the OCT three-dimensional image and output an image obtained by fusing the two-dimensional microscopic image from the right microscopic imaging module and the OCT three-dimensional image.

An OCT augmented reality-based surgical microscope imaging method uses the system as mentioned above to carry out imaging, and includes the following steps:

S1: adjusting the output intensity and focus positions of a surgical lighting unit and a guidance light source to enable cameras of a surgical microscope unit to clearly observe a surgical region and a guidance light spot, and acquiring a microscopic image of the surgical region;

S2: establishing a microscope two-dimensional Cartesian coordinate system Ox₀y₀ by taking a two-dimensional plane of the microscopic image acquired by the cameras as x and y axes, obtaining coordinates of the guidance light spot in the microscope coordinate system according to a position of the guidance light spot in the image, and using the obtained coordinates as a datum point; and changing, in an OCT three-dimensional scanning region, a deflection angle of a two-dimensional galvanometer scanner, acquiring coordinates of a series of different datum points to be marked as {A₁, A₂ . . . Aₙ};

S3: establishing a three-dimensional Cartesian coordinate system Ox₀y₀z₀, named an OCT coordinate system, by taking a plurality of pieces of continuous OCT slicing data at adjacent positions as volume data, taking an OCT depth scanning direction as a z axis and taking scanning directions of the two-dimensional galvanometer scanner as x and y axes; carrying out primary OCT three-dimensional scanning on an imaging region, wherein due to the fact that a scanner deflection angle corresponding to the projection position of guidance light in the step S2 is known, coordinate values of x₁ and y₁, corresponding to the position of the guidance light spot of the step S2, in the OCT coordinate system is also known, finding a boundary where the guidance light spot is located according to an OCT structure, thus acquiring coordinate values of z₁ of the guidance light spot of the step S2 in the OCT coordinate system, and finally, obtaining coordinates {B₁, B₂ . . . Bₙ} in the OCT coordinate system corresponding to the datum points {A₁, A₂ . . . Aₙ} in the microscope two-dimensional Cartesian coordinate system Ox₀y₀;

S4: carrying out fitting on {A₁, A₂ . . . Aₙ} and {B₁, B₂ . . . Bₙ} to obtain a transformation relationship from the OCT coordinate system to the microscope two-dimensional Cartesian coordinate system, which is a homography matrix corresponding to coordinate transformation, calibrating the cameras to obtain internal parameters of the cameras, and carrying out matrix operation to obtain external parameters of the cameras;

S5: adjusting the intensity of the surgical lighting unit, and simultaneously starting to carry out OCT three-dimensional scanning on the surgical region;

S6: setting virtual camera parameters of an OCT three-dimensional reconstructed portion according to the microscope external parameters obtained in the step S4 so as to obtain a registered OCT three-dimensional reconstructed image, and finally, carrying out superposition on the registered OCT three-dimensional reconstructed image and the microscopic image of the surgical region to complete virtual-and-real-image fusion display; and

S7: repeating the step S6 as OCT scanning continuously updates the input volume data, reconstructing all two-dimensional structural images to form a three-dimensional tomography model of the surgical region, and carrying out display by a display unit so as to implement real-time augmentation on the microscopic image of the surgical region.

Preferably, corresponding to the left camera and the right camera, respective microscope coordinate systems need to be respectively established, and then registration and fusion are carried out with an OCT image respectively.

Preferably, when the position of the datum point is set, the defection angle of the two-dimensional galvanometer scanner is a value during OCT three-dimensional scanning, instead of a random value in a scannable range.

Preferably, a number of the datum points required in the step S2 is n, n≥6.

The present disclosure has the beneficial effect that the surgical microscopic imaging system and method can accurately carry out registration and fusion on the two-dimensional microscopic image and the OCT three-dimensional image, thereby implementing real-time enhancement on the microscopic image of the surgical region, providing more intuitive navigation information for surgery, and implementing intuitive surgical guidance.

### BRIEF DESCRIPTION OF THE DRAWINGS

FIG. 1 is a schematic block diagram of configuration of an imaging system in accordance with an embodiment of the present disclosure;

FIG. 2 is a detail view of a structure of an imaging system in accordance with an embodiment of the present disclosure;

FIG. 3 is a flow chart of image fusion in accordance with an embodiment of the present disclosure;

FIG. 4 is a schematic diagram of establishment of a coordinate system and setting and search of a datum point in accordance with an embodiment of the present disclosure;

FIG. 5 shows a process and a result of fusion of a finger microscopic image and an OCT image in accordance with an embodiment of the present disclosure; and

FIG. 6 is a schematic diagram showing a spatial relationship of each coordinate system in accordance with an embodiment of the present disclosure.

### DESCRIPTION OF REFERENCE NUMERALS

1—surgical region; 2—objective lens; 3—OCT unit; 4—guidance light source; 5—light splitting unit; 6—optical zoom unit; 7—surgical microscope unit; 8—processing control unit; 9—display unit; 10—surgical lighting unit; 301—sweep-frequency laser; 302—first coupler; 303—wavelength division multiplexer; 304—first collimator; 305—two-dimensional galvanometer scanner; 306—second collimator; 307—reflector; 308—third collimator; 309—second coupler; 310—balance detector; 501—dichroic mirror; 701—left imaging lens; 702—right imaging lens; 703—left camera; and 704—right camera.

### DETAILED DESCRIPTION

The present disclosure will be further illustrated in detail below in combination with embodiments, so that those skilled in the art can implement accordingly with reference to the text of the specification.

It should be understood that terms such as “have”, “comprise” and “include” used herein are not exclusive of existence or addition of one or more other elements or a combination thereof

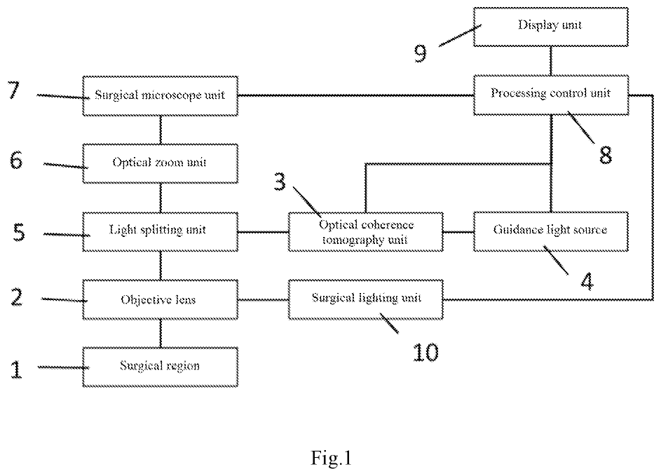

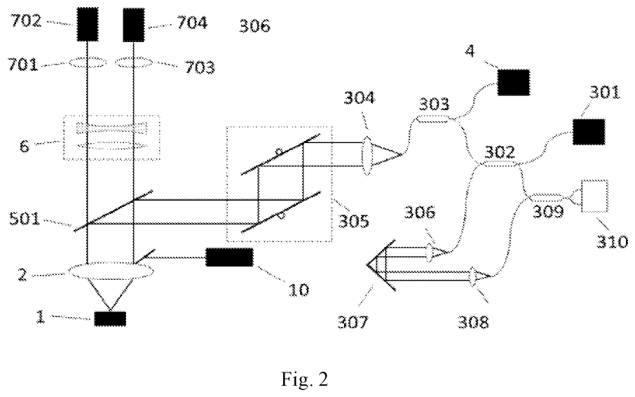

As shown in FIGS. 1-2, an OCT augmented reality-based surgical microscope imaging system in an embodiment includes:

a surgical microscope unit 7, configured to acquire a two-dimensional microscopic image of a surgical region 1;

an OCT unit 3, configured to acquire an OCT three-dimensional image of the surgical region 1;

a guidance light source 4, which can be captured by cameras of the surgical microscope unit 7 and is configured to project, into the surgical region, a guidance light spot synchronized with an OCT scanning light source of the OCT unit 3, light emitted by the guidance light source 4 being coaxial with OCT light;

a processing control unit 8, configured to acquire the two-dimensional microscopic image and the OCT three-dimensional image of the surgical region 1, and an image obtained by fusing the two-dimensional microscopic image and the OCT three-dimensional image of the surgical region 1; and

a display unit 9, configured to output and display a result of the processing control unit 8 to carry out navigation for surgery.

The system further includes a surgical lighting unit 10, and an objective lens 2, a light splitting unit 5 and an optical zoom unit 6 sequentially arranged along an imaging optical path of the surgical microscope unit 7;

the surgical lighting unit 10 is configured to provide lighting light for the surgical region 1, and the lighting light reflected by the surgical region 1 enters the surgical microscope unit 7 after sequentially passing through the objective lens 2, the light splitting unit 5 and the optical zoom unit 6 so as to implement two-dimensional microscopic imaging of the surgical region 1; and

light emitted by the guidance light source 4 and the OCT scanning light source of the OCT unit 3 reaches the surgical region 1 after sequentially passing through the light splitting unit 5 and the objective lens 2, and OCT scanning light reflected by the surgical region 1 backtracks to the OCT unit 3 to implement OCT three-dimensional imaging; and after guidance light reflected by the surgical region 1 passes through the light splitting unit 5, one portion of the guidance light enters the OCT unit 3, while the other portion of the guidance light enters the surgical microscope unit 7.

The surgical microscope unit 7 is configured to carry out two-dimensional imaging on the surgical region 1 via the objective lens 2, and the OCT unit 3 is configured to carry out two-dimensional scanning on the surgical region 1 via the objective lens 2 and implement three-dimensional tomography imaging of the surgical region 1 by the longitudinal tomography capacity of OCT. The surgical microscope unit 7 and the OCT unit 3 are configured to carry out coaxial imaging via an on-axis region of the objective lens 2. The guidance light source 4 is configured to project, into the surgical region 1, a guidance spot light source coaxial with the OCT scanning, and finally, can be captured by the cameras of the surgical microscope unit 7. The light splitting unit 5 is configured to implement light splitting and matching of light output by the microscope unit, the OCT unit 3 and the guidance light source 4 so as to implement coupling and separation of light with different wavelengths. The optical zoom unit 6 is configured for optical amplification of the surgical microscope unit 7 so as to achieve different imaging resolutions. The processing control unit 8 coordinates work of components, and acquires navigation information. The navigation information includes the two-dimensional microscopic image (a high-resolution surface microscopic result of the surgical region 1) and the OCT three-dimensional image (including an OCT three-dimensional imaging result of surgical instruments and tissue internal structures) of the surgical region 1, and an imaging result obtained by fusing the two-dimensional microscopic image and the OCT three-dimensional image. An output unit is a stereoscopic polarization optical display, and can output both a left path of navigation information and a right path of navigation information so as to carry out three-dimensional real-time monitoring on the surgical instruments and the tissues of the surgical region 1 in the surgery process. The surgical lighting unit carries out uniform lighting on the surgical region 1 via the objective lens 2.

The surgical microscope unit 7 is a binocular surgical microscope unit 7 and includes imaging lenses and cameras, the imaging lenses include a left imaging lens 701 and a right imaging lens 702, and the cameras include a left camera 703 and a right camera 704, wherein the left imaging lens 701 and the left camera 703 correspondingly constitute a left microscopic imaging module, and the right imaging lens 702 and the right camera 704 correspondingly constitute a right microscopic imaging module. The surgical microscope unit 7 is used for carrying out two-dimensional imaging on the surgical region 1 via the objective lens 2, and two-dimensional imaging is carried out on the surgical region 1 by the two cameras.

In the embodiment, the surgical microscope unit 7 is configured to carry out large-viewing-field two-dimensional imaging on a region where surgery is carried out, and the surgical region 1 can be, through the cameras, converted into a digital image which is displayed by the display unit 9. For example, light emitted by the surgical lighting unit 10 uniformly irradiates the surgical region 1 after passing through a rangefinder of the objective lens 2, and after being reflected by the surgical region 1, a lighting beam enters the surgical microscope unit 7 through a main axis of the objective lens 2, the light splitting unit 5 and the optical zoom unit 6, so that a surgery operator can directly observe, on the display unit 9, a binocular stereoscopic visual image obtained after image fusion of the surgical region 1.

In the embodiment, the OCT unit 3 is configured to carry out two-dimensional scanning on the surgical region 1 and obtain the three-dimensional image of the surgical region 1 by the longitudinal tomography capacity of the OCT technology. For example, an imaging beam of the OCT unit 3 transmits via the objective lens 2 to reach the surgical region 1, and after being reflected by the surgical region 1, the imaging beam passes through the objective lens 2 and the light splitting unit 5 and then returns to the OCT unit 3. The OCT unit 3 can convert a detected interference signal into an electrical signal, three-dimensional reconstruction is carried out in the processing control unit, and after registration is respectively carried out with double paths of viewing angles of a microscope, views of left and right eyes are acquired, so that the views are fused with the binocular image acquired by the surgical microscope unit 7. After processing, double-path output is carried out in the display unit 9, and the surgery operator can synchronously observe, in the display unit 9, the microscopic image with a stereoscopic perception effect and the OCT three-dimensional tomography image of the surgical region 1 so as to locate positions of the surgical instruments and the tissue internal structures in the three-dimensional space.

In a further preferred embodiment, the light splitting unit 5 is a dichroic mirror 501 which carries out total reflection on the light of the OCT unit 3, carries out semi-transmission and semi-reflection on the light of the guidance light source 4, and carries out total transmission on the light of the surgical lighting unit 10;

the OCT unit 3 includes the OCT scanning light source (which specifically is a sweep-frequency laser 301 in the embodiment), a first coupler 302, a wavelength division multiplexer 303, a first collimator 304, a two-dimensional galvanometer scanner 305, a second collimator 306, a reflector 307, a third collimator 308, a second coupler 309 and a balance detector 310;

an OCT scanning beam emitted by the OCT scanning light source is split into two paths of light via the first coupler 302, one path of light is sample light, and the other path of light is reference light;

guidance light emitted by the guidance light source 4 and the sample light are converged via the wavelength division multiplexer 303, then pass through the first collimator 304 together to become incident to the two-dimensional galvanometer scanner 305 to be deflected, and then are focused into the surgical region 1 by the objective lens 2 after being reflected by the dichroic mirror 501;

both the sample light and one portion of guidance light reflected by the surgical region 1 return along an original path after being reflected by the dichroic mirror 504, and reach one end of the second coupler 309 after passing through the first coupler 302; the other portion of guidance light reflected by the surgical region 1 transmits through the dichroic mirror 501 after passing through the objective lens 2, passes through the optical zoom unit 6, and then respectively passes through the left imaging lens 701 and the right imaging lens 702 to respectively enter the left camera 703 and the right camera 704;

the reference light emergent after passing through the first coupler 302 sequentially passes through the second collimator 306, the reflector 307 and the third collimator 308 to reach said one end of the second coupler 309, and enters the second coupler 309 together with the sample light and said one portion of guidance light that have been reflected by the surgical region 1 and reached said one end of the second coupler 309, and the reference light undergoes interference with the sample light and said one portion of guidance light before being received by the balance detector 310, and finally, a detection result is output to the processing control unit 8 so as to implement OCT three-dimensional imaging;

after a lighting beam emitted by the surgical lighting unit 10 irradiates the surgical region 1, the lighting light and the other portion of guidance light reflected by the surgical region 1 transmit through the dichroic mirror 501, then pass through the optical zoom unit 6 and subsequently enter the left microscopic imaging module and the right microscopic imaging module, and finally, an imaging signal is output to the processing control unit 8 so as to implement two-dimensional microscopic imaging of the surgical region 1; and

the processing control unit 8 carries out registration and fusion on the two-dimensional microscopic image and the OCT three-dimensional image of the surgical region 1, and a fused image is displayed and output by the display unit 9 so as to carry out navigation for surgery.

The display unit 9 is a polarized light display screen with a stereoscopic visual effect, and is configured to respectively output respective fused images of both the left visual pathway and right visual pathway (an image obtained by fusing the two-dimensional microscopic image from the left microscopic imaging module and the OCT three-dimensional image, and an image obtained by fusing the two-dimensional microscopic image from the right microscopic imaging module and the OCT three-dimensional image).

The guidance light source 4 and the lighting light unit may be controlled by the processing control unit, and the light intensity is controlled, so that the cameras can acquire the image, having the optimal effect, of the surgical region 1 as required, or can simultaneously distinguish the image of the surgical region 1 from the image of the guidance light spot.

The present disclosure further discloses an OCT augmented reality-based surgical microscope imaging method. The method uses the OCT augmented reality-based surgical microscope imaging system of the above-mentioned embodiments to carry out imaging.

A specific operation method for carrying out acquisition and fusion on a microscope image and an OCT three-dimensional image is as follows.

OCT signal processing includes: an interference signal acquired from a balance detector 310 is subjected to demodulation (including mean subtraction, windowing, inverse fast Fourier transform and mod value acquisition), and then intensity information of the interference signal in a depth domain is obtained. Then internal structure information of tissues of the surgical region 1 can be extracted according to surgery demands, wherein structural image mapping includes: logarithmic mapping, brightness, contrast mapping and 8-bit grey-scale map mapping. Based on the structural image, invalid information which influences the internal structure display, such as a nontransparent surface layer, is filtered out, valid surgical information, such as surgical instruments under tissues and target tissues, in an OCT image is reserved, and a new acquired image is used for input of subsequent three-dimensional reconstruction.

After being subjected to said processing, OCT original data is respectively fused with the images acquired by the left camera 703 and the right camera 704, wherein a to-be-fused OCT image needs to be registered in advance, and needs to be registered again each time when parameters related to surgical microscope navigation system imaging, such as the OCT scanning direction and the microscope imaging magnification, are changed.

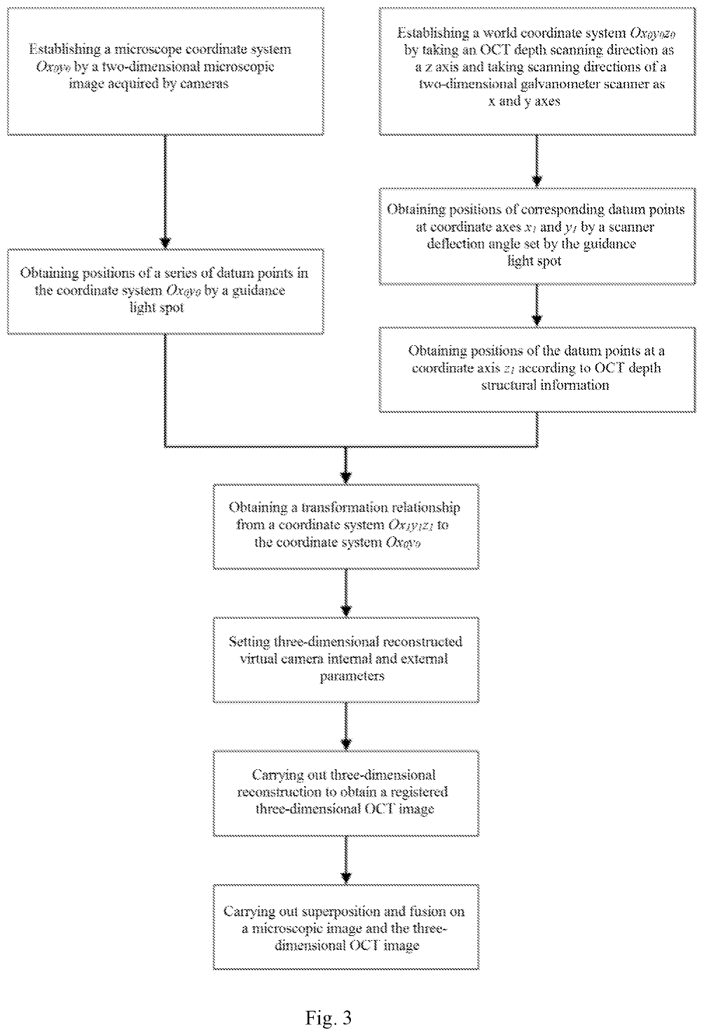

With reference to FIG. 3, the OCT augmented reality-based surgical microscope imaging method according to the embodiment includes the following steps:

S1: adjusting the output intensity and focus positions of a surgical lighting unit 10 and a guidance light source 4 to enable cameras of a surgical microscope unit 7 to clearly observe a surgical region 1 and a guidance light spot, and acquiring a microscopic image of the surgical region 1;

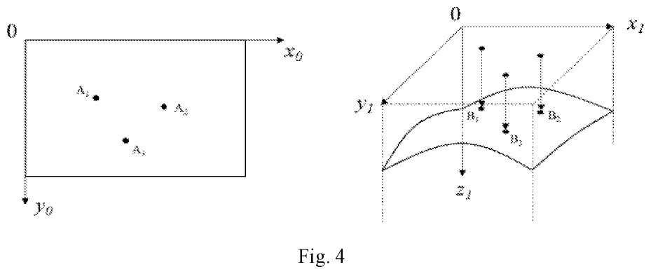

S2: establishing a microscope two-dimensional Cartesian coordinate system Ox₀y₀ by taking a two-dimensional plane of the microscopic image acquired by the cameras as x and y axes, obtaining coordinates of the guidance light spot in the microscope coordinate system according to a position of the guidance light spot in the image, and using the obtained coordinates as a datum point; and changing, in an OCT three-dimensional scanning region, a deflection angle of a two-dimensional galvanometer scanner, acquiring coordinates of a series of different datum points to be marked as {A1, A2 . . . An}, as shown in the left portion of FIG. 4 (only showing A1, A2 and A3);

S3: establishing a three-dimensional Cartesian coordinate system Ox₀y₀z₀, named an OCT coordinate system, by taking a plurality of pieces of continuous OCT slicing data at adjacent positions as volume data, taking an OCT depth scanning direction as a z axis and taking scanning directions of the two-dimensional galvanometer scanner as x and y axes; carrying out primary OCT three-dimensional scanning on an imaging region, wherein due to the fact that a scanner deflection angle corresponding to a projection position of guidance light in the step S2 is known, coordinate values of x₁ and y₁, corresponding to the position of the guidance light spot of the step S2, in the OCT coordinate system is also known, finding a boundary where the guidance light spot is located according to an OCT structure, thus acquiring coordinate values of z₁ of the guidance light spot of the step S2 in the OCT coordinate system, and finally, obtaining coordinates {B₁, B₂ . . . Bₙ} in the OCT coordinate system corresponding to the datum points {A₁, A₂ . . . Aₙ} in the microscope two-dimensional Cartesian coordinate system Ox₀y₀, as shown in the right portion of FIG. 4 (only showing B1, B2 and B3);

S4: carrying out fitting on {A₁, A₂ . . . Aₙ} and {B₁, B₂ . . . Bₙ} to obtain a transformation relationship from the OCT coordinate system to the microscope two-dimensional Cartesian coordinate system, which is a homography matrix corresponding to coordinate transformation, calibrating the cameras to obtain internal parameters of the cameras, and carrying out matrix operation to obtain external parameters of the cameras;

S5: adjusting the intensity of the surgical lighting unit 10 to the conventional surgical microscope imaging brightness, and simultaneously starting to carry out OCT three-dimensional scanning on the surgical region 1;

S6: setting virtual camera parameters of an OCT three-dimensional reconstructed portion according to the microscope external parameters obtained in the step S4 so as to obtain a registered OCT three-dimensional reconstructed image, and finally, carrying out superposition on the registered OCT three-dimensional reconstructed image and the microscopic image of the surgical region 1 to complete virtual-and-real-image fusion display; and

S7: repeating the step S6 as OCT scanning continuously updates the input volume data, reconstructing all two-dimensional structural images to form a three-dimensional tomography model of the surgical region 1, and carrying out display by a display unit 9 so as to implement real-time augmentation on the microscopic image of the surgical region 1.

When the above-mentioned steps are performed, corresponding to the left camera 703 and the right camera 704, respective microscope coordinate systems need to be respectively established, and then registration and fusion are carried out with an OCT image respectively so as to obtain an image fusion result with a binocular stereoscopic visual effect.

When the position of the datum point is set, the defection angle of the two-dimensional galvanometer scanner 305 is a value during OCT three-dimensional scanning, instead of a random value in a scannable range.

The above-mentioned steps need to be carried out again when the parameters related to system imaging (such as the OCT scanning direction and the microscope imaging magnification) are changed.

The OCT two-dimensional image participating in three-dimensional reconstruction only includes valid information, such as surgical instruments under tissues and target tissues, and cannot be shielded by invalid information above, such as a nontransparent tissue surface layer; and the image is extracted from the OCT two-dimensional structural image.

A number of the datum points required in the step S2 is n, n≥6.

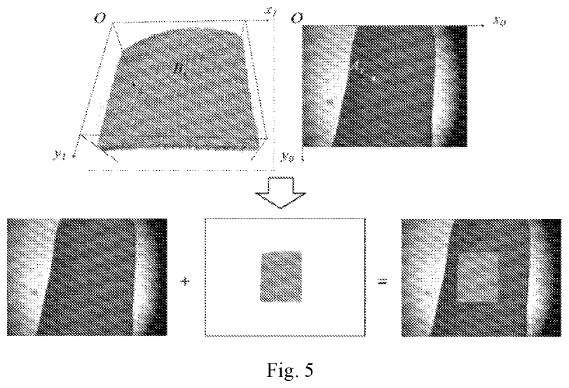

FIG. 5 shows a chart flow of fusion of a finger microscopic image and an OCT image acquired by single cameras, and in the process of acquiring the microscopic image, guidance light is turned on, and the galvanometer scanner is in a static state. The upper portion of FIG. 5 shows a three-dimensional OCT image in the OCT coordinate system from left to right and an image acquired by the camera in the microscope coordinate system, where Ai represents microscope coordinates of the guidance light spot, and Bi represents OCT coordinates of the guidance light spot. The lower portion of FIG. 5 shows a superposition process of the microscopic image and the registered three-dimensional OCT image, and shows a result after fusion.

The above-mentioned step S4 is the virtual-and-real registration process in augmented reality, and in an embodiment, the adopted specific principle and method are as follows:

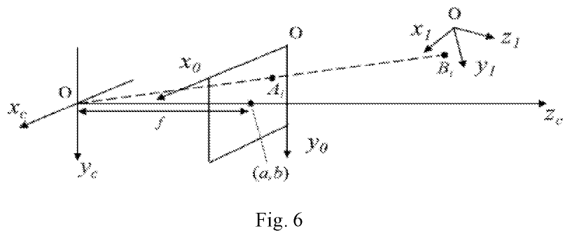

as shown in FIG. 4, Ox₁y₁z₁ is an OCT coordinate system and is used as a world coordinate system, i.e., an absolute coordinate system of the objective world; a three-dimensional Cartesian coordinate system Ox_{c}y_{c}z_{c} is a camera coordinate system, an origin is located at an optical center of a video camera, and z_{c} coincides with an optical axis; and Ox₀y₀ is a microscope coordinate system. With reference to FIG. 6, imaging transformation from Ox₁y₁z₁ to Ox₀y₀ can be described as follows:

a transformation relationship from the OCT coordinate system to the camera coordinate system is X_{c}:

$${X}_{c} = [ \begin{matrix}{x}_{c} \\ {y}_{c} \\ {z}_{c} \\ 1\end{matrix} ] = [ \begin{matrix}R & t \\ 0 & 1\end{matrix} ] [ \begin{matrix}{x}_{1} \\ {y}_{1} \\ {z}_{1} \\ 1\end{matrix} ] = {T}_{w} [ \begin{matrix}{x}_{1} \\ {y}_{1} \\ {z}_{1} \\ 1\end{matrix} ] \qquad ( 1 )$$

where R represents a rotation matrix in which rotation transformation is recorded, t represents a three-dimensional translation vector, and T_{w} includes a position and a direction of the camera relative to the world coordinate system, and thus are called as external parameters of the camera.

A transformation relationship from the camera coordinate system to the microscope coordinate system is Z₀:

$${Z}_{0} = [ \begin{matrix}{x}_{0} \\ {y}_{0} \\ 1\end{matrix} ] = [ \begin{matrix}\frac{1}{{d}_{x}} & 0 & a \\ 0 & \frac{1}{{d}_{y}} & b \\ 0 & 0 & 0\end{matrix} ] [ \begin{matrix}f & 0 & 0 & 0 \\ 0 & f & 0 & 0 \\ 0 & 0 & 1 & 0\end{matrix} ] [ \begin{matrix}{x}_{c} \\ {y}_{c} \\ {z}_{c} \\ 1\end{matrix} ] = [ \begin{matrix}{α}_{x} & 0 & a & 0 \\ 0 & {α}_{y} & b & 0 \\ 0 & 0 & 1 & 0\end{matrix} ] [ \begin{matrix}{x}_{c} \\ {y}_{c} \\ {z}_{c} \\ 1\end{matrix} ] = K [ \begin{matrix}{x}_{c} \\ {y}_{c} \\ {z}_{c} \\ 1\end{matrix} ] \qquad ( 2 )$$

where dx and dy represent physical distances of a pixel point of the microscope image on the x and y axes, f represents a distance from the microscope plane to the camera focal plane, a and b represent coordinates of a principal point of the camera in the microscope coordinate system, αₓ and α_{y} represent a height-to-width ratio of a pixel, αₓ=f/dx, and α_{y}=f/dy. K is only related to an internal structure of the camera, and thus is an internal parameter of the camera. A transformation relationship from the OCT coordinate system to the microscope coordinate system can be obtained from the formula (1) and the formula (2) as follows:

$$[ \begin{matrix}{x}_{0} \\ {y}_{0} \\ 1\end{matrix} ] = K {T}_{w} [ \begin{matrix}{x}_{1} \\ {y}_{1} \\ {z}_{1} \\ 1\end{matrix} ] = P [ \begin{matrix}{x}_{1} \\ {y}_{1} \\ {z}_{1} \\ 1\end{matrix} ] \qquad ( 3 )$$

For each pair of points Aᵢ and Bᵢ in the step S4, the following formula is met:

Aᵢ=PBᵢ, and P=KT_{w}=K[R|t] (4)

where, P represents a 3*4 matrix, and P can be solved through at least six pairs of Aᵢ and Bᵢ. P can be written as:

P=KT_{w}=K[R|t]=[KR|Kt]=[M|Kt] (5)

The rotation matrix R is an orthogonal matrix, and thus the following formula is met:

MM^{T}=KRR^{T}K^{T}=KK^{T} (6)

where the superscript T represents matrix transposition; and in addition, K represents an upper triangular matrix, and thus, K and R can be solved. Moreover, t can be obtained by the following formula:

t=K⁻¹(P₁₄P₂₄P₃₄)^{T} (7)

where the subscripts of P represent the matrix row and column. So far, the external parameters T_{w} and the internal parameter K of the camera all have been solved, i.e., the transformation relationship from the OCT coordinate system to the microscope two-dimensional Cartesian coordinate system is obtained.

The above three-dimensional reconstruction operation flow includes: inputting a plurality of continuous OCT slicing data at the adjacent positions as the volume data into the three-dimensional reconstructed portion, and based on a volume rendering algorithm, reconstructing all two-dimensional structural images to form the three-dimensional tomography model of the surgical region 1. The double-path image fusion result is finally output by a stereoscopic polarization optical display.

Although the implementation solution of the present disclosure has been disclosed as above, it is not limited to application listed in the specification and the implementation modes and it totally can be applicable to various fields suitable for the present disclosure. For those skilled in the art, additional modifications can be easily implemented, and thus, the present disclosure is not limited to the specific details without departure from the claims and the general concept defined by the equivalent range.

What is claimed is:

1. Aₙ optical coherence tomography (OCT) augmented reality-based surgical microscope imaging system, comprising:
a surgical microscope unit, configured to acquire a two-dimensional microscopic image of a surgical region;
an OCT unit, configured to acquire an OCT three-dimensional image of the surgical region;
a processing control unit, configured to acquire the two-dimensional microscopic image and the OCT three-dimensional image of the surgical region, and an image obtained by fusing the two-dimensional microscopic image and the OCT three-dimensional image of the surgical region;
a display unit, configured to output and display a result of the processing control unit to carry out navigation for surgery; and
a guidance light source, which can be captured by the surgical microscope unit and is configured to project, into the surgical region, a guidance light spot synchronized with an OCT scanning light source of the OCT unit.

2. The OCT augmented reality-based surgical microscope imaging system according to claim 1, further comprising a surgical lighting unit, and an objective lens, a light splitting unit and an optical zoom unit sequentially arranged along an imaging optical path of the surgical microscope unit, wherein
the surgical lighting unit is configured to provide lighting light for the surgical region, and the lighting light reflected by the surgical region enters the surgical microscope unit after sequentially passing through the objective lens, the light splitting unit and the optical zoom unit so as to implement two-dimensional microscopic imaging of the surgical region;
light emitted by the guidance light source and the OCT scanning light source of the OCT unit reaches the surgical region after sequentially passing through the light splitting unit and the objective lens, and OCT scanning light reflected by the surgical region backtracks to the OCT unit to implement OCT three-dimensional imaging; and after guidance light reflected by the surgical region passes through the light splitting unit, one portion of the guidance light enters the OCT unit, while the other portion of the guidance light enters the surgical microscope unit.

3. The OCT augmented reality-based surgical microscope imaging system according to claim 2, wherein the surgical microscope unit comprises imaging lenses and cameras, the imaging lenses include a left imaging lens and a right imaging lens, and the cameras include a left camera and a right camera, wherein the left imaging lens and the left camera correspondingly constitute a left microscopic imaging module, and the right imaging lens and the right camera correspondingly constitute a right microscopic imaging module.

4. The OCT augmented reality-based surgical microscope imaging system according to claim 3, wherein the light splitting unit is a dichroic mirror which carries out total reflection on the light of the OCT unit, carries out semi-transmission and semi-reflection on the light of the guidance light source, and carries out total transmission on the light of the surgical lighting unit.

5. The OCT augmented reality-based surgical microscope imaging system according to claim 1, wherein the OCT unit comprises the OCT scanning light source, a first coupler, a wavelength division multiplexer, a first collimator, a two-dimensional galvanometer scanner, a second collimator, a reflector, a third collimator, a second coupler and a balance detector;
an OCT scanning beam emitted by the OCT scanning light source is split into two paths of light via the first coupler, one path of light is sample light, and the other path of light is reference light;
guidance light emitted by the guidance light source and the sample light are converged via the wavelength division multiplexer, then pass through the first collimator together to become incident to the two-dimensional galvanometer scanner to be deflected, and then are focused into the surgical region by the objective lens after being reflected by the dichroic mirror;
both the sample light and one portion of guidance light reflected by the surgical region return along an original path after being reflected by the dichroic mirror, and reach one end of the second coupler after passing through the first coupler; the other portion of guidance light reflected by the surgical region transmits through the dichroic mirror after passing through the objective lens, passes through the optical zoom unit, and then respectively passes through the left imaging lens and the right imaging lens to respectively enter the left camera and the right camera;
the reference light emergent after passing through the first coupler sequentially passes through the second collimator, the reflector, and the third collimator to reach said one end of the second coupler, and enters the second coupler together with the sample light and said one portion of guidance light that have been reflected by the surgical region and reached said one end of the second coupler, and the reference light undergoes interference with the sample light and said one portion of guidance light before being received by the balance detector, and finally, a detection result is output to the processing control unit so as to implement OCT three-dimensional imaging;
after a lighting beam emitted by the surgical lighting unit irradiates the surgical region, the lighting light and the other portion of guidance light reflected by the surgical region transmit through the dichroic mirror, then pass through the optical zoom unit and subsequently enter the left microscopic imaging module and the right microscopic imaging module, and finally, an imaging signal is output to the processing control unit so as to implement two-dimensional microscopic imaging of the surgical region; and
the processing control unit carries out registration and fusion on the two-dimensional microscopic image and the OCT three-dimensional image of the surgical region, and a fused image is displayed and output by the display unit so as to carry out navigation for surgery.

6. The OCT augmented reality-based surgical microscope imaging system according to claim 5, wherein the display unit is a polarized light display screen with a stereoscopic visual effect, and is configured to respectively output an image obtained by fusing the two-dimensional microscopic image from the left microscopic imaging module and the OCT three-dimensional image and output an image obtained by fusing the two-dimensional microscopic image from the right microscopic imaging module and the OCT three-dimensional image.

7. Aₙ OCT augmented reality-based surgical microscope imaging method, using the system according to claim 2 to carry out imaging, and comprising the following steps:
S1: adjusting the output intensity and focus positions of a surgical lighting unit and a guidance light source to enable cameras of a surgical microscope unit to clearly observe a surgical region and a guidance light spot, and acquiring a microscopic image of the surgical region;
S2: establishing a microscope two-dimensional Cartesian coordinate system Ox₀y₀ by taking a two-dimensional plane of the microscopic image acquired by the cameras as x and y axes and taking the upper left corner of the microscopic image as an origin, obtaining coordinates of the guidance light spot in the microscope coordinate system according to a position of the guidance light spot in the image, and using the obtained coordinates as a datum point; and changing, in an OCT three-dimensional scanning region, a deflection angle of a two-dimensional galvanometer scanner, acquiring coordinates of a series of different datum points to be marked as {A₁, A₂ . . . Aₙ};
S3: establishing a three-dimensional Cartesian coordinate system Ox₀y₀z₀, named an OCT coordinate system, by taking a plurality of pieces of continuous OCT slicing data at adjacent positions as volume data, taking an OCT depth scanning direction as a z axis and taking scanning directions of the two-dimensional galvanometer scanner as x and y axes; carrying out primary OCT three-dimensional scanning on an imaging region, wherein, due to the fact that a scanner deflection angle corresponding to a projection position of guidance light in the step S2 is known, coordinate values of x₁ and y₁, corresponding to the position of the guidance light spot of the step S2, in the OCT coordinate system is also known, finding a boundary where the guidance light spot is located according to an OCT structure, thus acquiring coordinate values of z₁ of the guidance light spot of the step S2 in the OCT coordinate system, and finally, obtaining coordinates {B₁, B₂ . . . Bₙ} in the OCT coordinate system corresponding to the datum points {A₁, A₂ . . . Aₙ} in the microscope two-dimensional Cartesian coordinate system Ox₀y₀;
S4: carrying out fitting on {A₁, A₂ . . . Aₙ} and {B₁, B₂ . . . Bₙ} to obtain a transformation relationship from the OCT coordinate system to the microscope two-dimensional Cartesian coordinate system, which is a homography matrix corresponding to coordinate transformation, calibrating the cameras to obtain internal parameters of the cameras, and carrying out matrix operation to obtain external parameters of the cameras;
S5: adjusting the intensity of the surgical lighting unit, and simultaneously starting to carry out OCT three-dimensional scanning on the surgical region;
S6: setting virtual camera parameters of an OCT three-dimensional reconstructed portion according to the microscope external parameters obtained in the step S4 so as to obtain a registered OCT three-dimensional reconstructed image, and finally, carrying out superposition on the registered OCT three-dimensional reconstructed image and the microscopic image of the surgical region to complete virtual-and-real-image fusion display; and
S7: repeating the step S6 as OCT scanning continuously updates the input volume data, reconstructing all two-dimensional structural images to form a three-dimensional tomography model of the surgical region, and carrying out display by a display unit so as to implement real-time augmentation on the microscopic image of the surgical region.

8. The OCT augmented reality-based surgical microscope imaging method according to claim 7, comprising: establishing respective microscope coordinate systems corresponding to the left camera and the right camera respectively, and then respectively carrying out registration and fusion with an OCT image.

9. The OCT augmented reality-based surgical microscope imaging method according to claim 7, wherein when the position of the datum point is set, the defection angle of the two-dimensional galvanometer scanner is a value during OCT three-dimensional scanning, instead of a random value in a scannable range.

10. The OCT augmented reality-based surgical microscope imaging method according to claim 7, wherein a number of the datum points required in the step S2 is n, and n≥6.
